(19) United States
(12) Patent Application Publication
(10) Pub. No.: US 2024/0060993 A1
(43) Pub. Date: Feb. 22, 2024

(54) METHOD BY WHICH BIOSENSOR DEVICE DETECTS ANTIGEN BY USING MULTIPLE FREQUENCIES

(71) Applicant: BIOXONICS CO. LTD, Seoul (KR)

(72) Inventors: Kyeong Sik SHIN, Seoul (KR); Ji Yoon KANG, Seoul (KR)

(21) Appl. No.: 18/270,156

(22) PCT Filed: Dec. 28, 2021
(86) PCT No.: PCT/KR2021/020058

§ 371 Date: Jun. 28, 2023

(30) Foreign Application Priority Data
Dec. 31, 2020 (KR) 10-2020-0189197

(51) Int. Cl. G01N 33/68; G01N 33/543; G01N 27/02

(52) U.S. Cl. CPC G01N 33/6896; G01N 33/5438; G01N 33/54386; G01N 27/02; G01N 2333/4709; G01N 2800/2821

(57) ABSTRACT

Provided is a method by which a biosensor device detects an antigen by using multiple frequencies, the method enabling an antigen to be detected with high sensitivity. The method by which a biosensor device detects an antigen by using multiple frequencies includes: loading an antibody in the biosensor device including a micro-well that has an action electrode and a counterpart electrode disposed therein; measuring the impedance of the antibody for each of a first frequency and a second frequency; performing an antigen-antibody reaction by injecting an antigen into the biosensor device; using the first frequency and the second frequency to measure the impedances of each of the antigen and the antibody that are bound by means of the antigen-antibody reaction; and calculating changes in impedance that result from the antigen-antibody reaction from the impedances of the antigen and the antibody for the first frequency and the impedances of the antigen and the antibody for the second frequency.

### TECHNICAL FIELD

The technical idea of the present invention relates to a method of detecting an antigen using a sensor, and more particularly, to a method by which biosensor device detects antigen by using multiple frequencies.

### BACKGROUND ART

Recently, importance of technology for accurately detecting a trace of biological elements (antigens, antibodies, enzymes, etc.) using biosensor devices in the medical field has been gradually increased. Precise detection of biological factors using such a biosensor device is an essential element for accurate diagnosis and therapy of diseases.

For example, in order to accurately diagnose Alzheimer's disease commonly referred to as dementia, an accurate analysis of the cause of the Alzheimer's disease is required. The Alzheimer's disease is known to be caused by abnormally excessive generation and accumulation of amyloid beta in the brain. In order to identify the cause of the Alzheimer's disease, develop direct therapy methods and medicine, and further prevent the Alzheimer's disease through early diagnosis before the Alzheimer's disease develops, it is necessary to precisely detect and analyze the generation and accumulation of the amyloid beta, which is the cause of the disease. Of course, it is necessary to use a biosensor device having high sensitivity for accurate detection and analysis of such antigens.

### DISCLOSURE

### Technical Problem

A technical problem to be achieved by the technical idea of the present invention is to provide a method by which biosensor device detects antigen by using multiple frequencies that is capable of detecting antigens with high sensitivity.

However, these problems are exemplary, and the technical idea of the present invention is not limited thereto.

### Technical Solution

According to an aspect of the present invention, there is provided method by which biosensor device detects antigen by using multiple frequencies.

According to an embodiment of the present invention, the method by which biosensor device detects antigen by using multiple frequencies may include: loading an antibody into a biosensor device including a micro-well in which a working electrode and a counter electrode are disposed; measuring an impedance of the antibody with respect to each of a first frequency and a second frequency; performing an antigen-antibody reaction by injecting an antigen into the biosensor device; measuring an impedance of antigen-antibody linked by the antigen-antibody reaction using each of the first frequency and the second frequency; and calculating an impedance change according to the antigen-antibody reaction from the impedance of the antigen-antibody with respect to the first frequency and the impedance of the antigen-antibody with respect to the second frequency.

The calculating of the impedance change according to the antigen-antibody reaction may include: calculating a first normalized impedance by normalizing a first antigen-antibody impedance measured with respect to the first frequency of the antigen-antibody; calculating a second normalized impedance by normalizing a second antigen-antibody impedance measured with respect to the second frequency of the antigen-antibody; and calculating an impedance change value according to the antigen-antibody reaction using the first normalized impedance and the second normalized impedance.

The calculating of the first normalized impedance may be performed by dividing the first antigen-antibody impedance by a maximum value of a first antibody impedance measured using the first frequency for the antibody and normalizing the first antigen-antibody impedance.

The calculating of the second normalized impedance may be performed by dividing the second antigen-antibody impedance by a maximum value of a second antibody impedance measured using the second frequency for the antibody and normalizing the second antigen-antibody impedance.

The calculating of the impedance change value according to the antigen-antibody reaction using the first normalized impedance and the second normalized impedance may be performed by dividing the first normalized impedance by the second normalized impedance, dividing the second normalized impedance by the first normalized impedance, subtracting the second normalized impedance from the first normalized impedance, or subtracting the second normalized impedance from the first normalized impedance.

The performing of the antigen-antibody reaction and the measuring of the impedance of the antigen-antibody may be repeatedly performed while changing a concentration of the antigen.

The antigen detection method may further include, after the performing of the antigen-antibody reaction, performing cleaning to remove a non-specific conjugate using a cleaning solution.

The cleaning may be performed by moving the cleaning solution having phosphate buffer saline (PBS) containing 0.05 wt % to 0.2 wt % stearic acid ester of polyoxyethylene sorbitan.

The loading of the antibody may be performed by loading a bead to which the antibody is linked into the biosensor device.

The measuring of the impedance of the antibody may be performed under a flow of PBS.

In the measuring of the impedance of the antibody, the measurement of the impedance of the antibody using the first frequency and the measurement of the impedance of the antibody using the second frequency may be performed simultaneously or separately.

The measuring of the impedance of the antigen-antibody may be performed under a flow of PBS.

In the measuring of the impedance of the antigen-antibody, the measurement of the impedance of the antigen-antibody using the first frequency and the measurement of the impedance of the antigen-antibody using the second frequency may be performed simultaneously or separately.

The first frequency may range from 1 Hz to 100 Hz, and the second frequency may range from 500 Hz to 2,000 Hz.

The impedance of the antibody and the impedance of the antigen-antibody may be measured by applying a voltage ranging from 1 mV to 500 mV, or by applying a current ranging from 10 pA to 10 nA.

The impedance of the antibody and the impedance of the antigen-antibody may have the following relational expression.

$$Z = Rs + \frac{1 + {jwR}_{b} {C}_{b}}{{jwC}_{dl} - {w}^{2} {R}_{b} {C}_{b} {C}_{dl}}$$

(where, Z denotes the impedance, Rₛ denotes a solution resistance, R_{b} denotes a resistance between the bead and the electrode, C_{b} denotes a capacitance between the bead and the electrode, and C_{dl} denotes a capacitance between the electrodes)

The micro-well may include a substrate constituting a bottom surface, a working electrode and a counter electrode may be formed on one surface of the substrate, and a passivation layer forming a well-shaped inner space that isolates the antibody from an outside and accommodates the antibody may be provided on an upper portion of each of the working electrode and the counter electrode.

### Advantageous Effects

According to a method by which biosensor device detects antigen by using multiple frequencies according to the technical idea of the present invention, it is possible to measure an antigen concentration with high sensitivity by measuring an impedance using two or more frequencies.

The effects of the present invention described above have been described by way of example, and the scope of the present invention is not limited by these effects.

### DESCRIPTION OF DRAWINGS

FIG. 1 is a diagram illustrating a biosensor device using multiple frequencies according to an embodiment of the present invention.

FIG. 2 is a diagram schematically illustrating a biosensor unit performing a method by which biosensor device detects antigen by using multiple frequencies according to an embodiment of the present invention.

FIG. 3 is a flowchart illustrating a method by which biosensor device detects antigen by using multiple frequencies according to an embodiment of the present invention.

FIG. 4 is a schematic diagram illustrating a method by which biosensor device detects antigen by using multiple frequencies according to an embodiment of the present invention.

FIG. 5 is a detailed flowchart illustrating an operation of calculating an impedance change in the method by which biosensor device detects antigen by using multiple frequencies according to the embodiment of the present invention.

FIG. 6 is a diagram illustrating an impedance equivalent circuit used in the method by which biosensor device detects antigen by using multiple frequencies according to the embodiment of the present invention.

FIG. 7 is graphs showing an impedance change over time measured using the method by which biosensor device detects antigen by using multiple frequencies according to the embodiment of the present invention.

FIG. 8 is a graph showing a normalized impedance change over time measured using the method by which biosensor device detects antigen by using multiple frequencies according to the embodiment of the present invention.

FIG. 9 is a graph showing the relationship between the normalized impedance change and an antigen composition measured using the method by which biosensor device detects antigen by using multiple frequencies according to the embodiment of the present invention.

### BEST MODE

A method by which biosensor device detects antigen by using multiple frequencies according to an embodiment of the present invention includes: loading an antibody into a biosensor device including a micro-well in which a working electrode and a counter electrode are disposed; measuring an impedance of the antibody with respect to each of a first frequency and a second frequency; performing an antigen-antibody reaction by injecting an antigen into the biosensor device; measuring an impedance of antigen-antibody linked by the antigen-antibody reaction using each of the first frequency and the second frequency; and calculating an impedance change according to the antigen-antibody reaction from the impedance of the antigen-antibody with respect to the first frequency and the impedance of the antigen-antibody with respect to the second frequency.

### MODE FOR INVENTION

Hereinafter, exemplary embodiments of the present invention will be described in detail with reference to the accompanying drawings. Embodiments of the present invention will be provided only in order to further completely describe the technical idea of the present invention to those skilled in the art, the following exemplary embodiments may be modified into several other forms, and the scope of the technical idea of the present invention is not limited to the following exemplary embodiments. Rather, these exemplary embodiments make the present disclosure thorough and complete, and are provided to completely transfer the spirit of the present invention to those skilled in the art. Like reference numerals in this specification mean the same elements throughout. Further, various elements and regions in the drawings are schematically illustrated. Therefore, the spirit of the disclosure is not limited by relative sizes or intervals illustrated in the accompanying drawings.

An enzyme-linked immunosorbent assay (ELISA) is widely used in the field of modern biotechnology as an immunoassay. The enzyme-linked immunosorbent assay is a method of quantitatively measuring the strength and amount of an antigen-antibody reaction by linking an enzyme to an antigen or antibody and measuring activity of the enzyme.

However, the conventional enzyme-linked immunosorbent assay requires an antigen concentration of several tens of pg/ml or more, and therefore, is not suitable for detecting an antigen present in a very low concentration in a body fluid. Therefore, a more sensitive and precise detection method is required to detect an antigen, such as amyloid beta oligomer, present in a very low concentration in a body fluid.

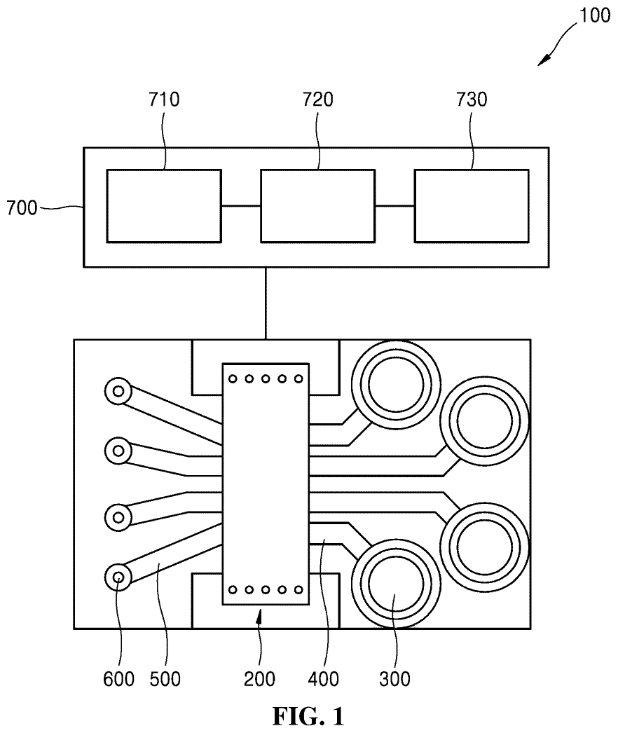

FIG. 1 is a diagram illustrating a biosensor device 100 using multiple frequencies according to an embodiment of the present invention.

Referring to FIG. 1, a biosensor device 100 includes a biosensor unit 200, a fluid injection unit 300, a fluid injection channel 400, a fluid ejection channel 500, and a fluid ejection unit 600.

The biosensor unit 200 is connected to the fluid injection unit 300 through the fluid injection channel 400 and connected to the fluid ejection unit 600 through the fluid ejection channel 500. A fluid containing an antibody or an antigen reaches the biosensor unit 200 through the fluid injection unit 300 and the fluid injection channel 400, and then the fluid is discharged to the outside through the fluid ejection channel 500 and the fluid ejection unit 600. Four fluid injection units 300, four fluid injection channels 400, four fluid ejection channels 500, and four fluid ejection units 600 are illustrated, but this is exemplary and the technical idea of the present invention is limited thereto.

The biosensor device 100 may further include a control unit 700 that is connected to the biosensor unit 200, inputs an electrical signal to the biosensor unit 200, and measures and calculates an electrical signal output from the biosensor unit 200. The control unit 700 may include an electrical signal input unit 710, an electrical signal measurement unit 720, and a calculation unit 730.

The biosensor device may be characterized in that it operates based on an electrochemical principle/mechanism.

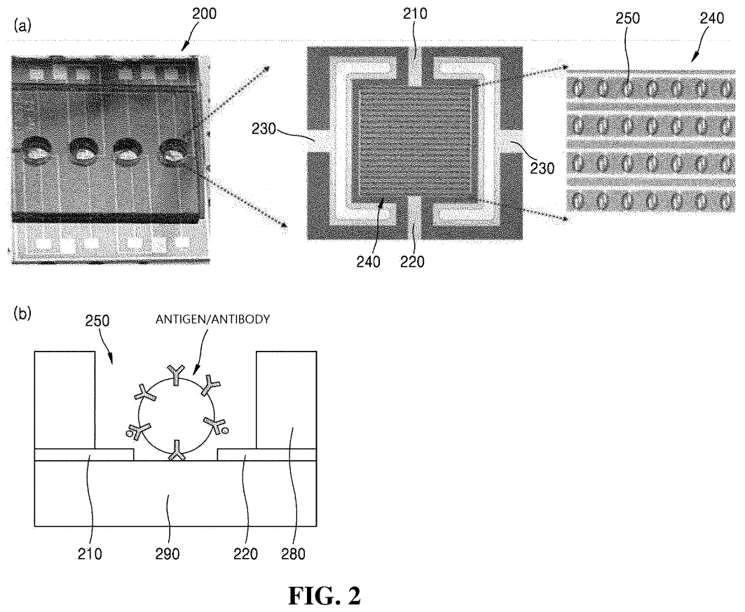

FIG. 2 is a diagram schematically illustrating a biosensor unit 200 performing a method by which biosensor device detects antigen by using multiple frequencies according to an embodiment of the present invention.

FIG. 2A illustrates the biosensor unit 200 enlarged in several stages, and FIG. 2B illustrates a state in which an antigen and an antibody are accommodated in the biosensor unit 200.

Referring to FIG. 2, the biosensor unit 200 is a biosensor for detecting an antigen, and includes a working electrode 210, a counter electrode 220, a reference electrode 230, and a micro-well array 240 that are disposed on a substrate 290. The structure of the biosensor unit 200 in FIG. 2 is merely for exemplifying the present invention, and the present invention is not limited thereto.

The substrate 290 constituting a bottom surface of the micro-well 250 is an electrically non-conductive material, and may include, for example, glass, polymer, or the like. The working electrode 210 and the counter electrode 220 are disposed on the substrate 290. The working electrode 210 and the counter electrode 220 may each include a conductive material, for example, a metal such as titanium or platinum. A passivation layer 280 is formed on each of the working electrode 210 and the counter electrode 220. The passivation layer 280 may serve as a structure such as a wall forming a well-shaped inner space that may isolate a magnetic support from the outside and accommodate the magnetic support. The passivation layer 280 is made of a material having electrical insulation properties, and may include, for example, an electrical insulation resin.

The substrate 290 may have a predetermined thickness and may be formed in a planar shape to support the working electrode 210, the counter electrode 220, the reference electrode 230, and the passivation layer 280.

The working electrode 210 and the counter electrode 220 may be electrically connected to the antigen and antibody. Accordingly, the amount of current may be measured by moving electrons generated from the antigen and antibody, or the impedance may be measured by applying an electric field to the antigen and antibody. These methods are capable of detecting minute amounts of antigen. To this end, the biosensor unit 200 includes a measurement unit that detects electrons generated in the micro-well as electrical signals or a measurement unit that may measure the impedance of the antigen and antibody.

The micro-well 250 defined by the passivation layer 280 may be formed. The micro-well 250 has a size suitable for accommodating the antigen and the antibody or the antibody, and may have a diameter ranging from 3 μm to 10 μm, for example. The micro-well 250 may have any of various shapes such as circular, elliptical, and polygonal shapes. Beads may be accommodated in the micro-well 250 using a magnetic substance and attached to the surface of the substrate 290. The micro-well 250 may accommodate one or more of the above antigens and antibodies, and confine electrons generated by an electric field and oxidation.

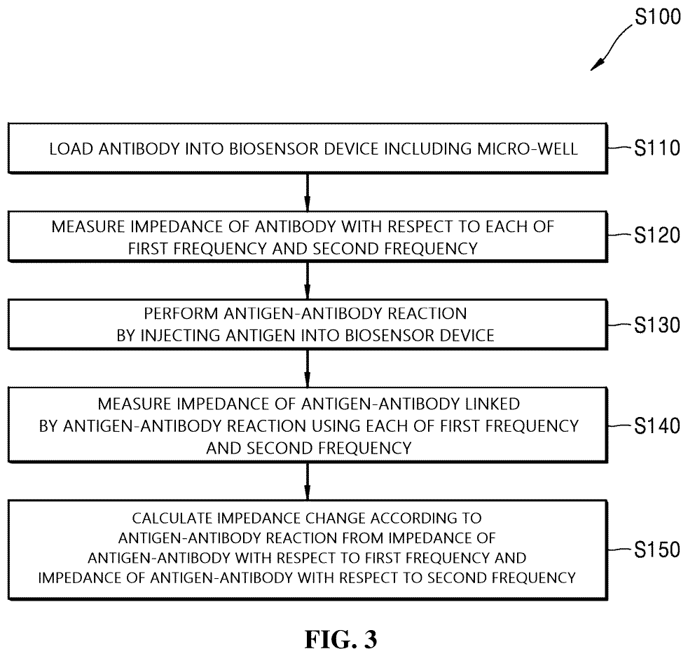

The plurality of micro-wells 250 are arranged in the micro-well array 240. The working electrode 210 and the counter electrode 220 are disposed on each micro-well 250. In the micro-well array 240 of FIG. 3, the micro-wells 250 having a diameter of 8 μm are arranged in 20 horizontal by 20 vertical. The amount of current may be increased by the micro-well array 240 and an error due to a positional deviation of the magnetic support 110 may be reduced. In addition, an electric field may be concentrated in the micro-well 250, so the antibody is localized to the attached bead, thereby improving detection sensitivity. In addition, when the micro-well 250 is formed, the diffusion of antigens and electrons generated by the reaction is limited by the wall of the micro-well 250, so it is possible to obtain the effect that the detection limit and signal sensitivity increase as the concentration of antigens and electrons between measuring electrodes increases.

The antigen may include various antigens. For example, in the case of checking Alzheimer's disease, amyloid beta oligomer may be included. The amyloid beta oligomer may include a recombinant Aβ42 oligomer. However, this is exemplary, and the case where the antigen includes various other substances is also included in the technical idea of the present invention. For example, various antigens such as a tau antigen (Tau), neurofilament light antigen, prostate-specific antigen (PSA), and a troponin antigen can be used. The solution containing the antigen may be a bodily fluid including blood, lymph, tissue fluid, and the like, and may include an intracellular fluid and an extracellular fluid.

The antigen may include various antigens. For example, in the case of checking Alzheimer's disease, an anti-Aβ antibody may be included. However, this is exemplary, and the case where the antigen includes various other substances is also included in the technical idea of the present invention. For example, the antibody may include various antibodies such as an anti-Tau antibody, an anti-Neurofilament light antibody, an anti-prostate-specific antigen antibody, and an anti-troponin antibody, depending on the antigen to be captured.

The antibody may be tagged with a peroxidase. The peroxidase serves to generate electrons by oxidizing an electron generator to be described below. The peroxidase may include, for example, horseradish peroxidase (HRP). The horseradish peroxidase is an enzyme found in a root of horseradish, widely used in biochemical applications, and a metal enzyme having many forms, and the most used type is a C type. A catalytic action is performed in an oxidation reaction of many organic substrates by the contained hydrogen peroxide. However, this is exemplary, and the cases in which the peroxidase includes various materials are also included in the technical idea of the present invention. For example, NADH peroxidase, flavoprotein oxidases, manganese peroxidase, lignin peroxidase, and the like may be used as the peroxidase.

When measuring an impedance by the antigen-antibody reaction using the biosensor device 100, an undesirable impedance response of nonlinear characteristics may appear. The reason is analyzed as follows. First, phenomena such as charge accumulation occur in magnetic beads according to the continuous application of electrical signals. Second, when the position of the bead is not completely fixed, the flow of the fluid makes noise and the like. Third, since electricity is continuously applied to the fluid, characteristics of the fluid may change according to changes in local temperature. Electrical characteristics due to these causes generate nonlinear characteristics or signal drift, and thus signal extraction is difficult. Therefore, in order to overcome this problem, the antigen detection method using the biosensor device 100 according to the technical idea of the present invention measures impedance using multiple frequencies.

FIG. 3 is a flowchart illustrating an antigen detection method S100 of a biosensor device using multiple frequencies according to an embodiment of the present invention.

Referring to FIG. 3, the antigen detection method S100 of a biosensor device using multiple frequencies includes: loading an antibody into a biosensor device including a micro-well in which a working electrode and a counter electrode are arranged (S110); measuring an impedance of the antibody with respect to each of a first frequency and a second frequency (S120); performing an antigen-antibody reaction by injecting an antigen into the biosensor device (S130); measuring an impedance of antigen-antibody linked by the antigen-antibody reaction using each of the first frequency and the second frequency (S140); and calculating an impedance change according to the antigen-antibody reaction from the impedance of the antigen-antibody with respect to the first frequency and the impedance of the antigen-antibody with respect to the second frequency (S150).

In the measuring of the impedance of the antibody (S120) and the measuring of the impedance of antigen-antibody (S140), an electrical signal is input from the electrical signal input unit 710 of the control unit 700 to the biosensor unit 200, and the impedance as an electrical signal to be measured accordingly may be measured by the electrical signal measurement unit 720 of the control unit 700. The calculating of the impedance change (S150) may be performed by a calculation unit 730 of the control unit 700. However, the configuration of the control unit 700 is exemplary and the technical idea of the present invention is not limited thereto.

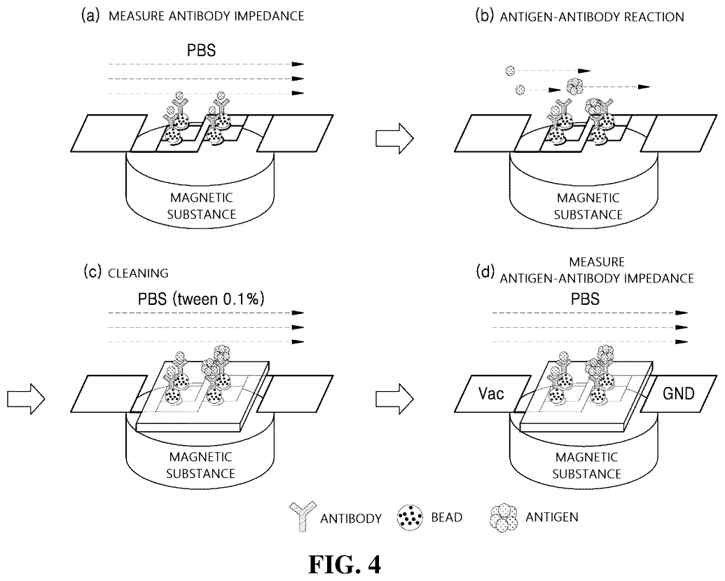

FIG. 4 is a schematic diagram illustrating the antigen detection method S100 of a biosensor device using multiple frequencies according to an embodiment of the present invention.

Referring to FIGS. 3 and 4A, the loading of the antibody into the biosensor device including the micro-well (S110) and the measuring of the impedance of the antibody for each of a first frequency and a second frequency (S120), are performed.

The loading of the antibody (S110) is performed by loading the bead to which the antibody is linked into the biosensor device.

The bead is a structure for attaching the antibody to its surface and is made of a material having magnetism or a material capable of being magnetized. For example, the bead may include a metal including iron, cobalt, nickel, or the like or a synthetic material. The bead may have any of various sizes, for example, a diameter ranging from about 1 μm to 4 μm, and for example, a diameter of 2.8 μm. The bead may be loaded into the micro-well of the biosensor device by a magnetic substance disposed below.

The measuring of the impedance of the antibody (S120) may be performed under a flow of phosphate buffer saline (PBS).

The measuring of the impedance of the antibody (S120) may be performed by applying a voltage ranging from 1 mV to 500 mV, or by applying a current ranging from 10 pA to 10 nA.

An electrical signal applied to the antibody may have a plurality of frequencies, for example, the first frequency and the second frequency. The impedance for the first frequency and the impedance for the second frequency may be separated and extracted using a filter. The first frequency may be a low frequency, for example, in the range of 1 Hz to 100 Hz, and for example, 50 Hz. The second frequency may be a high frequency higher than the first frequency, for example, in the range of 500 Hz to 2000 Hz, and for example, 1000 Hz.

In the measuring of the impedance of the antibody (S120), the measurement of the impedance of the antibody using the first frequency and the measurement of the impedance of the antibody using the second frequency may be performed simultaneously or separately.

The impedance may be measured using various devices, and for example, the impedance may be measured by cyclic voltammogram (CV) and amperometric measurement.

Referring to FIGS. 3 and 4B, the injecting of the antigen into the biosensor device to perform the antigen-antibody reaction (S130) is performed.

The antigen-antibody reaction is performed by moving an antigen having a specific concentration over the bead to which the antibody is linked. The antigen may be linked to the antibody, and the antibody and the antigen may be linked to and positioned on the bead. Even in the operation S130 of performing the antigen-antibody reaction, the impedance measurement using the first frequency and the second frequency may be continuously performed.

Referring to FIGS. 3 and 4C, after the performing the antigen-antibody reaction (S130), a cleaning operation of removing non-specific conjugate using a cleaning solution is performed. The cleaning operation may be performed by moving the cleaning solution having PBS containing 0.05 wt % to 0.2 wt % stearic acid ester of polyoxyethylene sorbitan (tween). The cleaning operation may be optional and omitted. Even in the cleaning operation, the impedance measurement using the first frequency and the second frequency may be continuously performed.

Referring to FIGS. 3 and 4D, the measuring of the impedance of the antigen-antibody (S140) linked by the antigen-antibody reaction using each of the first frequency and the second frequency is performed.

The measuring of the impedance of the antigen-antibody (S140) may be performed under the flow of the PBS.

The measuring of the impedance of the antigen-antibody (S140) may be performed by applying a voltage ranging from 1 mV to 500 mV, or by applying a current ranging from 10 pA to 10 nA.

An electrical signal applied to the antigen-antibody may have a plurality of frequencies, for example, the first frequency and the second frequency. The impedance for the first frequency and the impedance for the second frequency may be separated and extracted using a filter. The first frequency may be a low frequency, for example, in the range of 1 Hz to 100 Hz, and for example, 50 Hz. The second frequency may be a high frequency higher than the first frequency, for example, in the range of 500 Hz to 2000 Hz, and for example, 1000 Hz.

In the measuring of the impedance of the antigen-antibody (S140), the measurement of the impedance of the antigen-antibody using the first frequency and the measurement of the impedance of the antigen-antibody using the second frequency may be performed simultaneously or separately.

The performing of the antigen-antibody reaction (S130) and the measuring of the impedance of the antigen-antibody (S140) may be repeatedly performed while changing a concentration of the antigen.

For example, the concentration of the antigen may be varied in the range of 1 pg/ml to 100 pg/ml. For example, the concentration of the antigen may be changed stepwise to 1 pg/ml, 10 pg/ml, and 100 pg/ml.

Referring back to FIG. 3, the calculating of the impedance change according to the antigen-antibody reaction from the impedance of the antigen-antibody for the first frequency and the impedance of the antigen-antibody for the second frequency (S150) is performed.

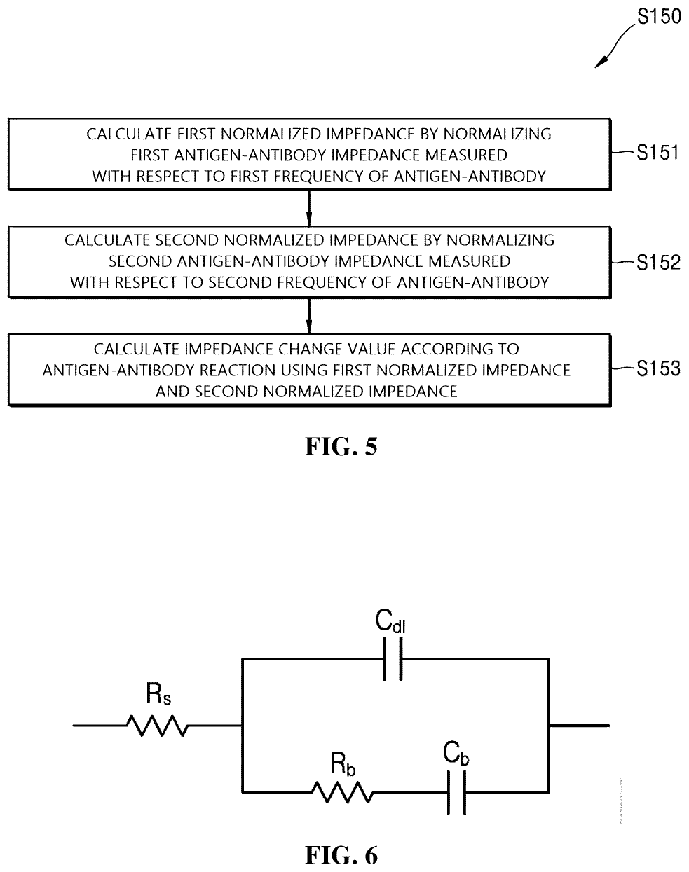

FIG. 5 is a detailed flowchart illustrating the calculating of the impedance change (S150) in the method by which biosensor device detects antigen by using multiple frequencies (S100) according to the embodiment of the present invention.

Referring to FIG. 5, the calculating of the impedance change (S150) includes calculating a first normalized impedance by normalizing a first antigen-antibody impedance measured with respect to the first frequency of the antigen-antibody (S151), calculating a second normalized impedance by normalizing a second antigen-antibody impedance measured with respect to the second frequency of the antigen-antibody (S152), calculating an impedance change value according to the antigen-antibody reaction using the first normalized impedance and the second normalized impedance (S153).

The calculating of the first normalized impedance (S151) may be performed by dividing the first antigen-antibody impedance by a maximum value of a first antibody impedance measured using the first frequency for the antibody and normalizing the first antigen-antibody impedance.

The calculating of the second normalized impedance (S152) may be performed by dividing the second antigen-antibody impedance by a maximum value of a second antibody impedance measured using the second frequency for the antibody and normalizing the second antigen-antibody impedance.

The calculating of the impedance change value according to the antigen-antibody reaction using the first normalized impedance and the second normalized impedance (S153) may be performed by dividing the first normalized impedance by the second normalized impedance, dividing the second normalized impedance by the first normalized impedance, subtracting the second normalized impedance from the first normalized impedance, or subtracting the second normalized impedance from the first normalized impedance.

FIG. 6 is a diagram illustrating an impedance equivalent circuit used in the method by which biosensor device detects antigen by using multiple frequencies according to the embodiment of the present invention.

Referring to FIG. 6, an impedance equivalent circuit of the antibody or the antigen-antibody is illustrated. In addition, the impedance of the antibody and the impedance of the antigen-antibody may have the following relational expression.

$$Z = Rs + \frac{1 + {jwR}_{b} {C}_{b}}{{jwC}_{dl} - {w}^{2} {R}_{b} {C}_{b} {C}_{dl}}$$

(where, Z denotes the impedance, Rₛ denotes a solution resistance, R_{b} denotes a resistance between the bead and the electrode, C_{b} denotes a capacitance between the bead and the electrode, and C_{dl} denotes a capacitance between the electrodes)

The impedance Z has a frequency dependent relationship. When the resistance R_{b} between the bead and the electrode, the capacitance C_{b} between the bead and the electrode, and the capacitance C_{dl} between the electrodes change due to the change in the concentration of the antigen in the solution, it can be seen that the impedance value itself having frequency dependence changes.

Accordingly, the resistance R_{b}, the capacitance C_{b}, and the capacitance C_{dl} in the initial condition without the antigen and the degree of change when the circuit components change as the antigen-antibody reaction occurs due to the occurrence of the antigen-antibody reaction may vary depending on frequency, and thus, the change aspect of the impedance is changed.

### Experimental Example

Hereinafter, exemplary Experimental Examples are presented to help the understanding of the present invention. However, the following experimental examples are only for help understanding of the present invention, and the present invention is not limited by the following Experimental Examples.

An experimental example was performed using the method by which biosensor device detects antigen by using multiple frequencies according to the technical idea of the present invention.

The impedance was measured as described above while performing the antigen-antibody reaction on the antibody and the antigen in the biosensor device. The antibody used was primary antibody 6E10, and the antigen used was amyloid beta.

For the impedance measurement, a voltage of 20 mV was applied, and a frequency of 50 Hz as the first frequency and a frequency of 1000 Hz as the second frequency were used. The concentration of antigen changed to 1 pg/ml, 10 pg/ml, and 100 pg/ml.

The time interval was divided as follows.
First period: T₀ to T₁, the antibody reacted with the bead to be linked to the bead, and the impedances for the two frequencies were measured while the PBS solution was flowing. Note that there is no antigen in this period.
Second period: T₁ to T₂, and the impedances for the two frequencies were measured as the antigen-antibody reaction was generated by injecting an antigen solution having a first concentration of 1 pg/ml.
Third period: T₂ to T₃, and the impedances for the two frequencies were measured while the cleaning solution was flowing.
Fourth period: T₃ to T₄, and the impedances for the two frequencies were measured while the PBS solution was flowing. The impedance of the fourth period becomes first impedance S₁ for the antigen-antibody generated by the antigen solution having the first concentration of 1 pg/ml. The first impedance S₁ is an average value of the impedances measured in the fourth period or a value obtained by subtracting the minimum value from the maximum value.
Fifth period: T₄ to T₅, and the impedances for the two frequencies were measured as the antigen-antibody reaction was generated by injecting an antigen solution having a second concentration of 10 pg/ml.
Sixth period: T₅ to T₆, and the impedances for the two frequencies were measured while the PBS solution was flowing. The impedance of the sixth period becomes second impedance S₁₀ for the antigen-antibody generated by the antigen solution having the second concentration of 10 pg/ml. The second impedance S₁₀ is an average value of the impedances measured in the sixth period or a value obtained by subtracting the minimum value from the maximum value.
Seventh period: T₆ to T₇, and the impedances for the two frequencies were measured while the antigen-antibody reaction was generated by injecting an antigen solution having a third concentration of 100 pg/ml.
Eighth period: T₇ to T₆, and the impedances for the two frequencies were measured while the PBS solution was flowing. The impedance of the eighth period becomes second impedance S₁₀₀ for the antigen-antibody generated by the antigen solution having the third concentration of 100 pg/ml. The third impedance S₁₀₀ is an average value of the impedances measured in the eighth period or a value obtained by subtracting the minimum value from the maximum value.

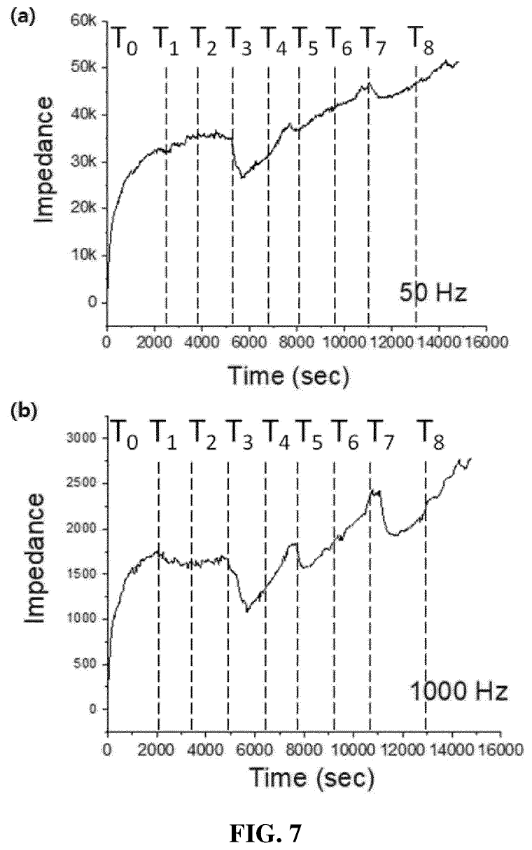

FIG. 7 is graphs showing an impedance change over time measured using the method by which biosensor device detects antigen by using multiple frequencies according to the embodiment of the present invention.

FIG. 7A is a case of a first frequency of 50 Hz, and FIG. 7B is a case of a second frequency of 1000 Hz. The impedance changes for the first to eighth periods were shown.

For each frequency, the normalization was performed using the following formula:

Normalized impedance value of first frequency=|Z1|(f1)/|Z1|max(f1)

Normalized impedance value of second frequency=|Z2|(f2)/|Z2|max(f2)

Here, “|Z1|max(f1)” denotes the maximum impedance value for the first frequency in the first period, and |Z1|(f1) is an impedance value for the first frequency in the first to eighth periods. In addition, “|Z2|max(f2)” denotes the maximum impedance value for the second frequency in the first period, and |Z2|(f2) denotes an impedance value for the second frequency in the first to eighth periods.

Subsequently, the normalized impedance change over time may be obtained by the “normalized impedance value of the first frequency/normalized impedance value of the second frequency.”

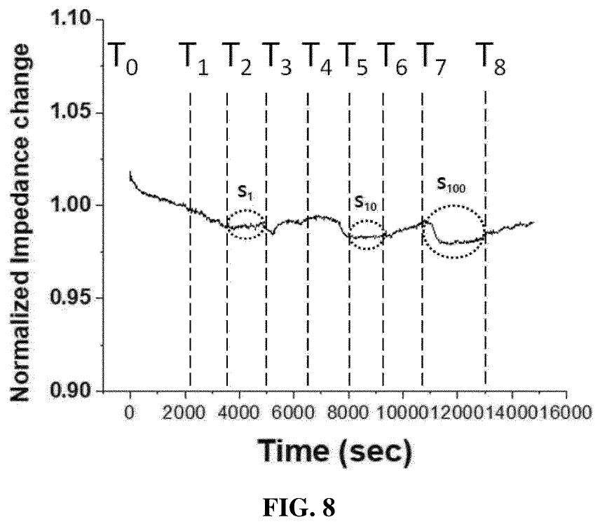

FIG. 8 is a graph showing a normalized impedance change over time measured using the method by which biosensor device detects antigen by using multiple frequencies according to the embodiment of the present invention.

Referring to FIG. 8, the normalized impedance change of each concentration may be found from the normalized impedance change over time. In the above graph, the first normalized impedance change corresponding to the first concentration of 1 pg/ml was indicated as S₁, the second normalized impedance change corresponding to the second concentration of 10 pg/ml was indicated as S₁₀, and the third normalized impedance change of 100 pg/ml was indicated as S₁₀₀.

The first normalized impedance change S₁ appeared in the third period T₂ to T₃, the second normalized impedance change S₁₀ appeared in the sixth period T₅ to T₆, and the third normalized impedance change S₁₀₀ appeared in the eighth period T₇ to T₈.

Then, by calculating (1−S₁)×100, (1−S₁₀)×100, and (1−S₁₀₀)×100, the correlation between the concentration of the antigen and the change in impedance can be found. Since the reference signal was set to 1, the normalized impedance change was subtracted from 1.

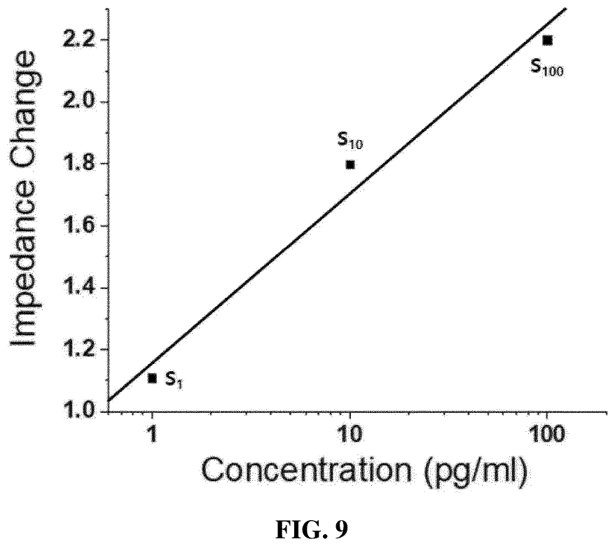

FIG. 9 is a graph showing the relationship between the normalized impedance change and an antigen composition measured using the method by which biosensor device detects antigen by using multiple frequencies according to the embodiment of the present invention.

Referring to FIG. 9, it can be seen that the normalized impedance change increases as the antigen composition increases, and the relationship can be seen that the normalized impedance change increases linearly with respect to the log value of the antigen composition.

It will be obvious to those skilled in the art to which the present invention pertains that the technical idea of the present invention described above is not limited to the above-described exemplary embodiments and the accompanying drawings, but may be variously substituted, modified, and altered without departing from the scope and spirit of the present invention.

<Reference Signs List>

| | | |
|---|---|---|
| 100: Biosensor device | | |
| 200: Biosensor unit | 210: Working electrode | 220: Counter |
| | | electrode |
| 230: Reference electrode | 240: Micro-well array | |
| 250: Micro-well | 280: Passivation layer | 290: Substrate |
| 300: Fluid injection unit | 400: Fluid injection channel | |
| 500: Fluid ejection channel | 600: Fluid ejection unit | |

### INDUSTRIAL APPLICABILITY

A method by which biosensor device detects antigen by using multiple frequencies according to the technical idea of the present invention has industrial applicability capable of measuring an antigen concentration with high sensitivity by measuring an impedance using two or more frequencies.

1. A method by which biosensor device detects antigen by using multiple frequencies, comprising:
loading an antibody into a biosensor device including a micro-well;
measuring an impedance of the antibody with respect to each of a first frequency and a second frequency;
performing an antigen-antibody reaction by injecting an antigen into the biosensor device;
measuring an impedance of antigen-antibody linked by the antigen-antibody reaction using each of the first frequency and the second frequency; and
calculating an impedance change according to the antigen-antibody reaction from the impedance of the antigen-antibody with respect to the first frequency and the impedance of the antigen-antibody with respect to the second frequency.

2. The antigen detection method of claim 1, wherein the calculating of the impedance change according to the antigen-antibody reaction includes:
calculating a first normalized impedance by normalizing a first antigen-antibody impedance measured with respect to the first frequency of the antigen-antibody;
calculating a second normalized impedance by normalizing a second antigen-antibody impedance measured with respect to the second frequency of the antigen-antibody; and
calculating an impedance change value according to the antigen-antibody reaction using the first normalized impedance and the second normalized impedance.

3. The antigen detection method of claim 2, wherein the calculating of the first normalized impedance is performed by dividing the first antigen-antibody impedance by a maximum value of a first antibody impedance measured using the first frequency for the antibody and normalizing the first antigen-antibody impedance.

4. The antigen detection method of claim 2, wherein the calculating of the second normalized impedance is performed by dividing the second antigen-antibody impedance by a maximum value of a second antibody impedance measured using the second frequency for the antibody and normalizing the second antigen-antibody impedance.

5. The antigen detection method of claim 2, wherein the calculating of the impedance change value according to the antigen-antibody reaction using the first normalized impedance and the second normalized impedance is performed by:
dividing the first normalized impedance by the second normalized impedance;
dividing the second normalized impedance by the first normalized impedance;
subtracting the second normalized impedance from the first normalized impedance; or
subtracting the second normalized impedance from the first normalized impedance.

6. The antigen detection method of claim 1, wherein the performing of the antigen-antibody reaction and the measuring of the impedance of the antigen-antibody are repeatedly performed while changing a concentration of the antigen.

7. The antigen detection method of claim 1, further comprising, after the performing of the antigen-antibody reaction, performing cleaning to remove a non-specific conjugate using a cleaning solution.

8. The antigen detection method of claim 7, wherein the cleaning is performed by moving the cleaning solution having phosphate buffer saline (PBS) containing 0.05 wt % to 0.2 wt % stearic acid ester of polyoxyethylene sorbitan.

9. The antigen detection method of claim 1, wherein the loading of the antibody is performed by loading a bead to which the antibody is linked into the biosensor device.

10. The antigen detection method of claim 1, wherein the measuring of the impedance of the antibody is performed under a flow of phosphate buffer saline (PBS).

11. The antigen detection method of claim 1, wherein, in the measuring of the impedance of the antibody, the measurement of the impedance of the antibody using the first frequency and the measurement of the impedance of the antibody using the second frequency are performed simultaneously or separately.

12. The antigen detection method of claim 1, wherein the measuring of the impedance of the antigen-antibody is performed under a flow of phosphate buffer saline (PBS).

13. The antigen detection method of claim 1, wherein, in the measuring of the impedance of the antigen-antibody, the measurement of the impedance of the antigen-antibody using the first frequency and the measurement of the impedance of the antigen-antibody using the second frequency are performed simultaneously or separately.

14. The antigen detection method of claim 1, wherein the first frequency ranges from 1 Hz to 100 Hz, and
the second frequency ranges from 500 Hz to 2,000 Hz.

15. The antigen detection method of claim 1, wherein the impedance of the antibody and the impedance of the antigen-antibody are measured by applying a voltage ranging from 1 mV to 500 mV, or by applying a current ranging from 10 pA to 10 nA.

16. The antigen detection method of claim 1, wherein the impedance of the antibody and the impedance of the antigen-antibody have the following relational expression,
$$Z = Rs + \frac{1 + {jwR}_{b} {C}_{b}}{{jwC}_{dl} - {w}^{2} {R}_{b} {C}_{b} {C}_{dl}} ,$$
(where, Z denotes the impedance, Rₛ denotes a solution resistance, R_{b} denotes a resistance between the bead and the electrode, C_{b} denotes a capacitance between the bead and the electrode, and C_{dl} denotes a capacitance between the electrodes).

17. The antigen detection method of claim 1, wherein the micro-well includes a substrate constituting a bottom surface,
a working electrode and a counter electrode are formed on one surface of the substrate, and
a passivation layer forming a well-shaped inner space that isolates the antibody from an outside and accommodates the antibody is provided on an upper portion of each of the working electrode and the counter electrode.
